(19) United States
(12) Patent Application Publication
(10) Pub. No.: US 2012/0057007 A1
(43) Pub. Date: Mar. 8, 2012

(54) Simplified Visual Screening Check on Television

(76) Inventor: Satoshi Ishiguro, San Diego, CA (US)

(21) Appl. No.: 12/875,192

(22) Filed: Sep. 3, 2010

(51) Int. Cl. H04N 7/18; A61B 3/02

(52) U.S. Cl. 348/61; 351/237; 351/246; 348/E07.085; 348/563; 348/734

(57) ABSTRACT

A television based visual screening method involves sequentially presenting a plurality of numbers of varying size on a television display; receiving a plurality of signals from a remote controller as a result of a user input from a keypad of the remote controller; determining if the signals received from the remote controller match the displayed numbers; deriving a measure of the user's vision from the matching of the received signals with the displayed numbers; and displaying a result indicative of the measure of the user's vision from the measure. This abstract is not to be considered limiting, since other embodiments may deviate from the features described in this abstract.

### COPYRIGHT AND TRADEMARK NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever. Trademarks are the property of their respective owners.

### BACKGROUND

Early detection of problems with vision can be a factor in avoiding developmental difficulties in children and detecting visual changes in both children and adults that should be attended to by a trained medical professional.

### BRIEF DESCRIPTION OF THE DRAWINGS

Certain illustrative embodiments illustrating organization and method of operation, together with objects and advantages may be best understood by reference detailed description that follows taken in conjunction with the accompanying drawings in which:

FIG. 1 is an example of a television cross media bar menu system incorporating a visual screening test function consistent with certain embodiments of the present invention.

FIG. 2 is an example of use of a remote controller to control a visual screening test consistent with certain embodiments of the present invention.

FIG. 3 is an example grid array of numbers useful in one implementation consistent with certain embodiments of the present invention.

FIG. 4 is an example flow chart of a process consistent with certain implementations consistent with certain embodiments of the present invention.

FIG. 5 is another example flow chart of one implementation of a visual screening test consistent with certain embodiments of the present invention.

FIG. 6 is another more detailed example flow chart of one implementation of a visual screening test consistent with certain embodiments of the present invention.

FIG. 7 is another example flow chart of a visual screening test consistent with certain embodiments of the present invention.

FIG. 8 is an example television receiver device consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically. The term “program” or “computer program” or similar terms, as used herein, is defined as a sequence of instructions designed for execution on a computer system. A “program”, or “computer program”, may include a subroutine, a function, a procedure, an object method, an object implementation, in an executable application, an applet, a servlet, a source code, an object code, a shared library/dynamic load library and/or other sequence of instructions designed for execution on a computer system. The term “processor”, “controller”, “CPU”, “Computer” and the like as used herein encompasses both hard programmed, special purpose, general purpose and programmable devices and may encompass a plurality of such devices or a single device in either a distributed or centralized configuration without limitation.

Reference throughout this document to “one embodiment”, “certain embodiments”, “an embodiment”, “an example”, “an implementation” or similar terms means that a particular feature, structure, or characteristic described in connection with the embodiment, example or implementation is included in at least one embodiment, example or implementation of the present invention. Thus, the appearances of such phrases or in various places throughout this specification are not necessarily all referring to the same embodiment, example or implementation. Furthermore, the particular features, structures, or characteristics may be combined in any suitable manner in one or more embodiments, examples or implementations without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

In accord with certain implementations consistent with the present embodiment, a simplified vision screening check can be carried out on a TV screen at home. The TV can be programmed to show a vision check screen, and the user can check their vision by responding to the TV by using the TV remote controller keypad. Of course, is should be understood that embodiments consistent with the present invention are not intended to be a substitute for diagnosis by a trained medical professional, but can nonetheless be useful as a screening test to determine that the advice of a professional may be in order.

For purposes of this document, numbers are considered to be of similar size if each of the numbers has the same height in number of pixels from its uppermost point to its lowermost point, or if each of the numbers can be considered to be of the same number of “points”. In this case, the number of points is determined in the manner that a point would be used in determination of a font size.

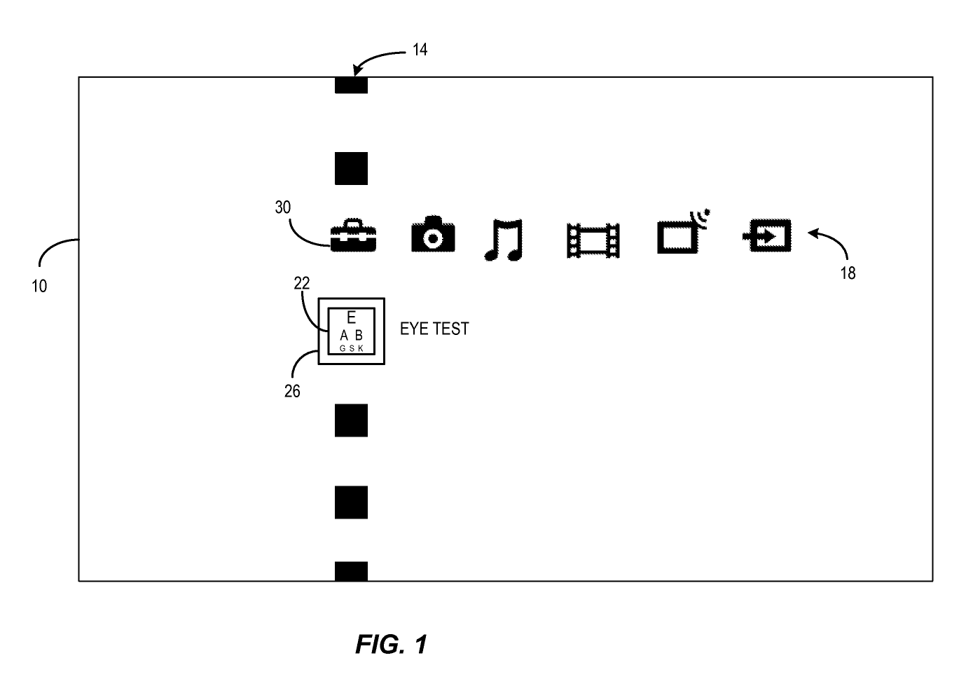

Turning now to FIG. 1, an example television menu system, shown as a cross-media bar (XMB) menu system is depicted. In this illustration, the display 10 shows a vertical bar of menu selections 14 that intersects a horizontal bar 18 and in this example, one of the vertical bar menu selections 22 is depicted as an eye test screen which may appear highlighted as 26 (depicted in this figure by the box enclosing the selection) as a function under the toolbox selection 30. The user can access such a cross media bar menu selection on certain of the Sony Bravia® series of television sets by pressing the <home> or <menu> key of the remote controller and then using navigation keys (up, down, left, right) to arrive at the desired menu selection.

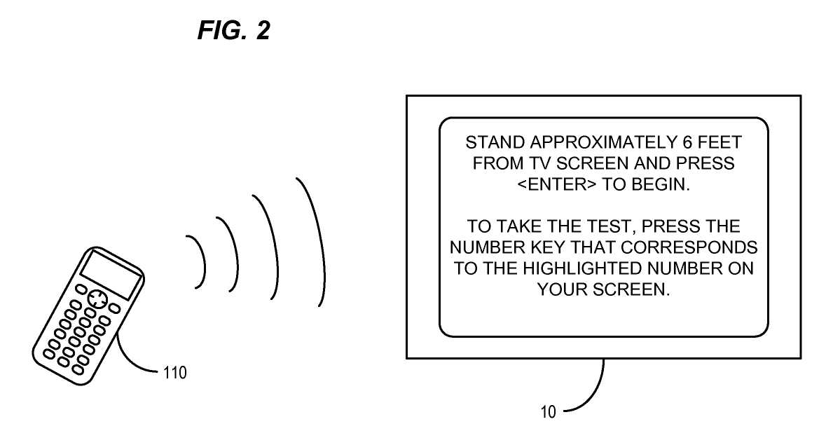

Turning to FIG. 2, a remote controller 110 is used to make the visual screen selection by appropriate navigation by the user. In an alternative embodiment, the function may be implemented by direct entry of a vision test command from the remote controller 110. Upon entry of the vision test command by whatever mechanism, the user may be prompted on the TV display 10 with basic instructions for taking the vision test. In this example, the user is instructed to position himself or herself at a specified distance from the TV display and press the <enter> key to begin. The instructions, for this implementation, also tell the user to press the numerical key on the remote controller 110 keypad that corresponds to the highlighted number on the television display.

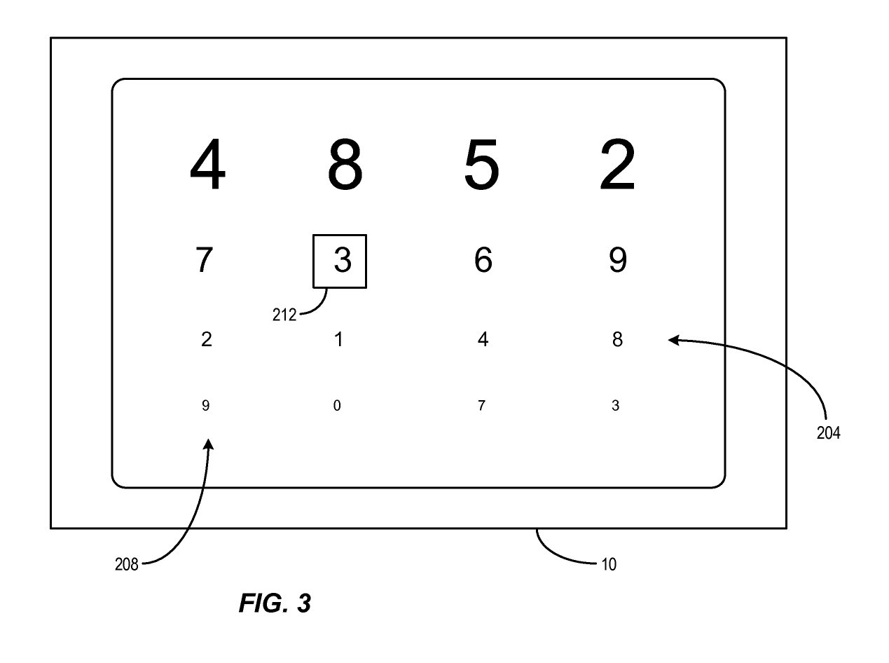

In this implementation, upon entering the visual screening program from the prior display, the user is presented with a display screen such as that shown in FIG. 3 having a grid-like arrangement of numbers of varying size. In this illustrative example, the grid is made up of rows 204 and columns 208 and at this point in the test, the number 3 is displayed as highlighted either by a box 212 as shown, or by other highlighting means such as color background, flashing or the like. In this example, the user would respond correctly in this portion of the test by pressing the <3> key on the remote controller 110 numerical keypad. The program running on the television receiver device then proceeds to present by highlighting another number. In one implementation, the bottom row of numbers is small and get progressively larger as the rows of numbers approach the top of the display 10. In one example, the program may start with the smallest row of numbers at the bottom and check to see if the user can read and correctly respond to the prompts (highlighting) and if all are correct, testing with the larger numbers is unnecessary and the program may terminate at this point and indicate that the user's visual acuity is good.

It will be clear upon consideration of the present teachings that the actual size of the numbers displayed is determined by the visual acuity associated with viewing that size of numbers at a specified distance in the same manner used for generation of conventional Snellen eye chart character sizes. However, accuracy may be limited somewhat by the achievable contrast and limited number of characters available as those associated with the remote controller numerical keypad. Accuracy can be improved by use of a more extensive alpha-numeric keypad on the remote controller with the possible accompanying tradeoff of remote controller size.

In another implementation, the test program may start at the top row and work down to lower rows until the user begins to make errors indicative that the limits of the user's visual acuity has been reached. The program may then terminate at this point with an indication of the user's visual acuity. In other implementations, various other procedures including random presentation of the numbers can be used without limitation.

Note that all numbers need not be arranged in rows where each row is a different size. In another implementation, the size of the numbers can be arranged so that each column represents numbers of a different size. In yet another implementation, the numbers of varying sizes can be arranged randomly on the display. In any such arrangement, the collection of numbers may be referred to as an array without regard for whether the arrangement is in the form of rows and columns In other implementations, numbers of varying sizes can be displayed as a single number on the display for a user to match.

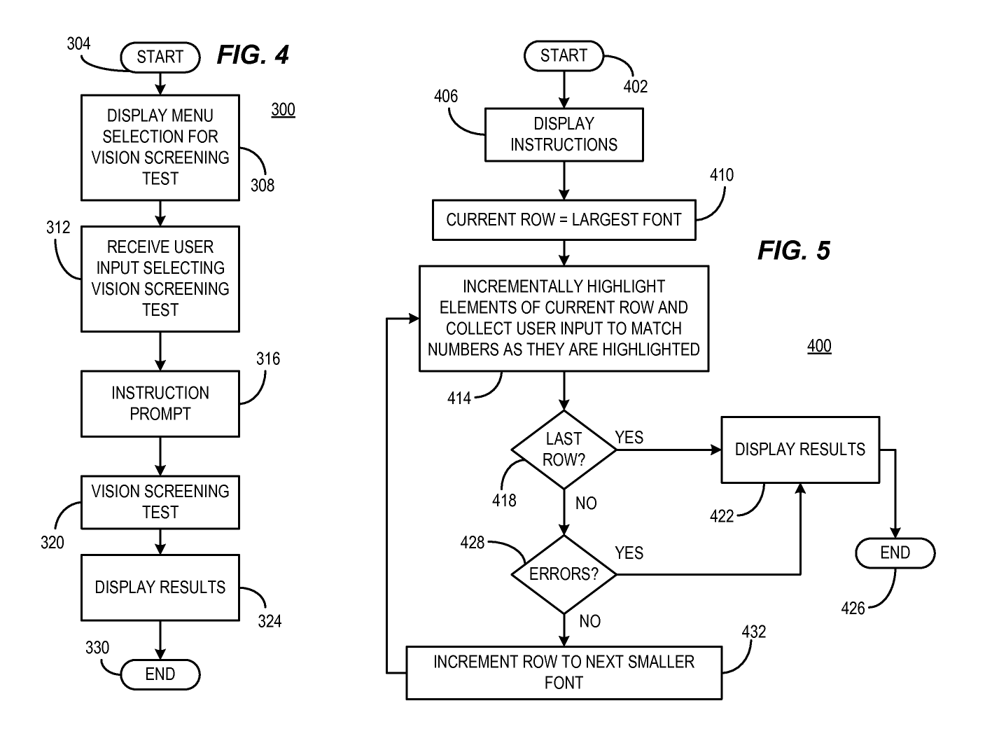

One example process as described above is depicted in FIG. 4 as process 300 starting at 304. At 308 a menu is displayed to permit the user to select a visual screening test. The user selects the test at 312 by use of appropriate interaction with a remote controller 110. After this, the user may be prompted with instructions at 316 after which the vision screening test is carried out at 320 and the results of the test are displayed at 324 and the process ends at 330. The displayed results may be in many forms including conventional visual acuity test terms such as “you vision is approximately 20/30” or in other terms such as how well the user's performance score or an analysis thereof. On example of the latter results might be “You missed three out of four of the numbers on line four—a person with 20/20 vision or better would be expected to get all of the numbers on line 4 correct—you got all of the numbers on line 3 correct—a person with 20/30 vision or better would be expected to get all of the numbers on line 4 correct”. In another implementation, the user can simply be given the test results such as:

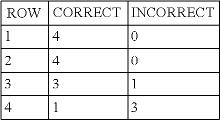

With such raw data, the user can compare his or her vision with friends and family and empirically determine whether or not there is a visual problem. Of course, those skilled in the art will appreciate upon consideration of the present teachings that it is generally not practical to have a television display situated great distances from the user in a typical home room. But, the results at, for example 6 feet (or other distance which can be fixed or user selectable), can be extrapolated to a conventional measurement of visual acuity. Those skilled in the art will further appreciate upon consideration of the present teachings that users of such a test should be aware that it is only a screening test that can be impacted by many factors and is not intended as an absolutely accurate diagnostic tool.

Referring to FIG. 5, a process 400 starting at 402. At 406, instructions are displayed after the user selects the visual screening test using any suitable method.

At 410, the current row is initialized at the row containing the largest numbers. At 414, the elements of the current row are sequentially highlighted to prompt the user to enter each number. In an alternative implementation, the entire row may be highlighted and the user enters all numbers—possibly followed by <enter>. In either event, the user input is collected at 414 as it is received from the remote controller and matched and an analysis of the results is performed. When the last row of user input has been received at 418, the results as determined by the analysis is displayed at 422 and the process ends at 426. In certain implementations, the process may terminate prematurely if a sufficient number of errors are encountered on a particular row at 428 and control passes from 428 to 422. Otherwise, at 428, the row is incremented to the next row of smaller numbers at 432. In this manner, once the limits of the user's visual acuity has been reached, it is pointless to continue testing. While preferred implementations display multiple rows from which numbers are highlighted, it will be appreciated that a single row or a single number of a particular size can be displayed individually for each iteration of the test without limitation.

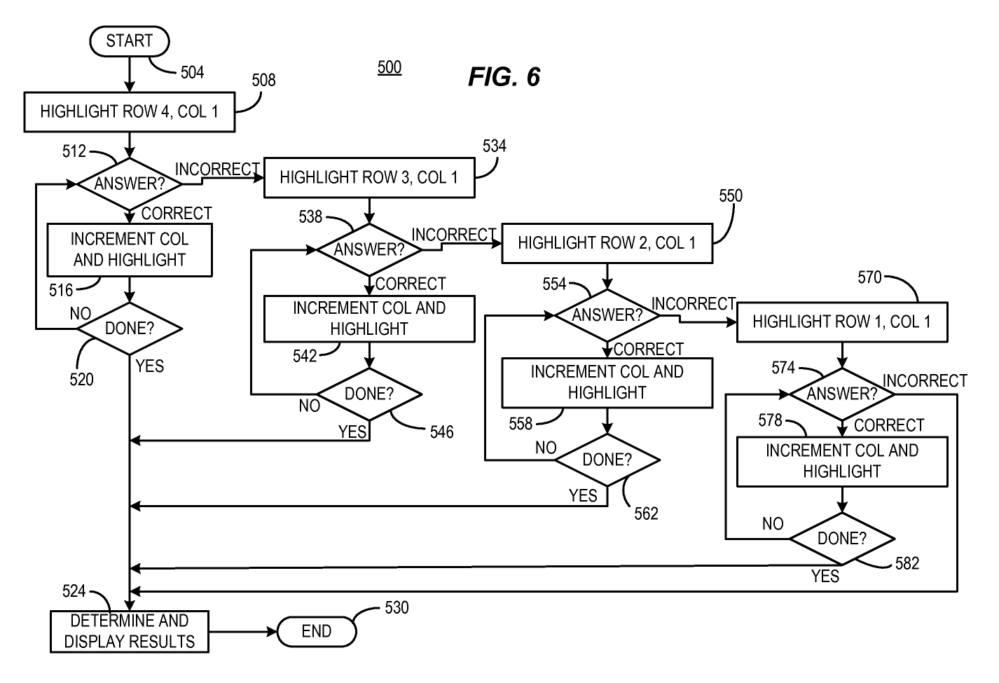

FIG. 6 depicts a more detailed example process 500 wherein a single incorrect answer on a row triggers movement to a larger font row. In this example, a chart similar to that shown in FIG. 3 is assumed where the rows are numbered 1-4 with the smallest font numbers being on the 4^{th} row as shown. This process 500 starts at 504 and the number at the first column of row 4 is highlighted at 508 indicating that the user is being queried to enter the number at this coordinate in the row-column array. The user answers with an input from the keypad of the remote controller at 512 and a determination is made as to whether the answer is correct or incorrect at 512. So long as the answers are correct, the column is incremented to highlight the next number at 516 and control passes to 512 to await the answer. When the last column is reached without error at 520, there is no need to test with larger font ant the results are determined and displayed at 524. If an incorrect answer is given at 512, the process increments rows by passing control to 534 where the number at position row 3, column 1 is highlighted.

The process entered at 534 operates in much the same way as the process used for row 4 with answers being checked at 538 and the columns being incremented and the next number highlighted at 542 until the row is done at 546 in which case control again passes to 524 to determine and display the results. A single incorrect answer, however, passes the process to the next row (row 2) starting at 550.

The process entered at 550 operates in much the same way as the process used for rows 3 and 4 with answers being checked at 554 and the columns being incremented and the next number highlighted at 558 until the row is done at 562 in which case control again passes to 524 to determine and display the results. A single incorrect answer, however, passes the process to the next row (row 1) starting at 570. In this example, four rows are used, but this process can be carried out for any number of rows with new rows scrolling into place if necessary.

The process entered at 570 operates in much the same way as the process used for rows 2, 3 and 4 with answers being checked at 574 and the columns being incremented and the next number highlighted at 578 until the row is done at 582 in which case control again passes to 524 to determine and display the results. A single incorrect answer, however, passes the process directly to 524 where the conclusion is that the user's vision is worse than the visual acuity associated with the first row.

As noted above, however, the measurement could proceed in the same manner with more rows either initially present or scrolled into place to further attempt to approximate the user's vision.

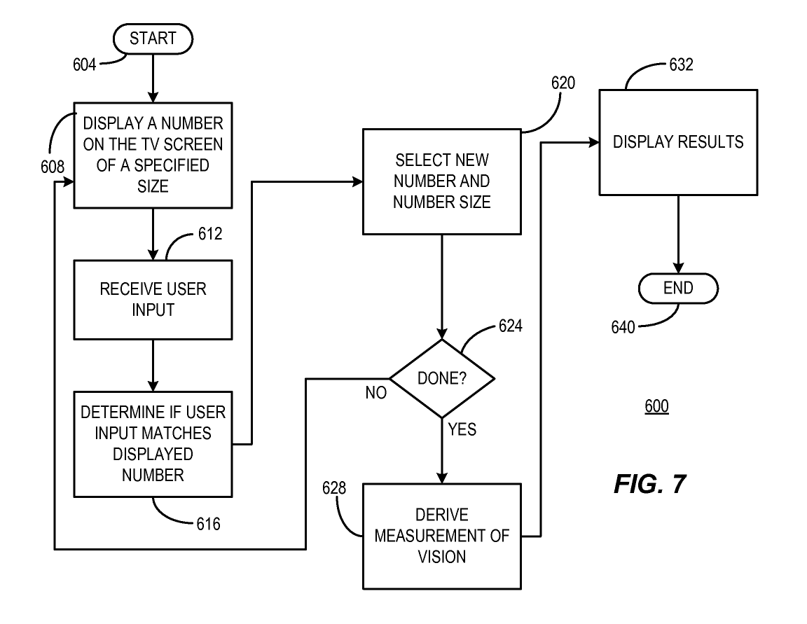

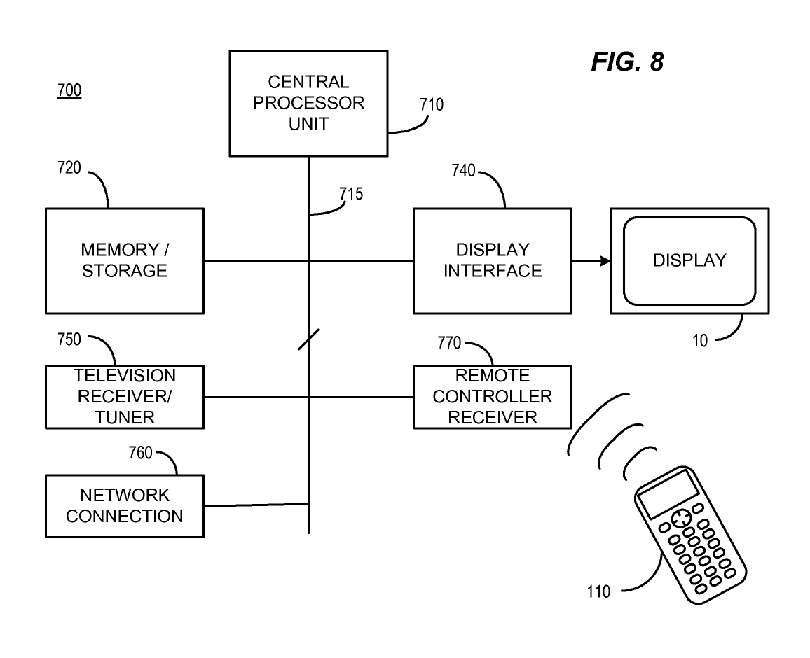

A variation of the embodiments discussed above can be implemented wherein a sequence of numbers of varying sizes can be displayed—where in this case, displaying the numbers is itself considered the way the numbers are presented to the user in much the same way that highlighting is used in the other examples, but a single number is displayed at any given time in the process 600 depicted in FIG. 7 starting at 604. At 608, a number is displayed on the television display 10 with the number having a known approximate size. The user then provides input from the remote controller keypad at 612 and at 616, the process determines if the user input matches the displayed number. The process then selects a new number and number size at 620 for display on the TV screen at 608 and the process repeats until the processer determines that the process is done at 624. At this point, when the process is complete, a measurement of vision is derived at 628 and the results are displayed at 632. The process ends at 640.

Any of the above processes and variations thereof can be carried out on a television receiver device such as a television set or set top box using a programmed processor or hardware equivalent. One basic implementation is depicted in FIG. 8 as television receiver device 700. Device 700 has one or more central processor units (CPU) 710 with an associated bus 715 (or multiple buses) used to connect the central processor unit 710 to memory and storage represented as 720 in a known manner. A display interface 740 is provided to either supply an output for an external display or directly interfaced to an integral display 10. The television device 700 includes a television receiver/tuner 750 for receiving television signals from an over the air broadcast or cable or satellite source. In some instances, television device 700 may also or alternatively include a network connection 760 for receiving television programming via the Internet, for example using Sony Corporation's Bravia™ Internet Video Link (BIVL™) which may be coupled to a local area network (LAN) and/or wide area network (WAN) and/or the Internet. In order to receive signals such as infrared (IR) or radio frequency (RF) remote controller signals, a remote controller receiver 770 is interfaced to the system so that the user can initiate commands to start the visual screening test and to carry out the test function using a keypad of the remote controller 110 as shown.

Thus, a visual screening method according to certain implementations involves on a television display, displaying an array of numbers arranged in rows and columns of numbers, where each of the rows contains numbers of a similar size and where the size of the numbers in each row differs from the size of numbers in each of the other rows; sequentially highlighting a plurality of the numbers displayed on the television display in order to prompt a user input; for each of the sequentially highlighted numbers, receiving a signal from a remote controller as a result of a user input to a number pad of the remote controller; determining if each of the received signals matches the displayed numbers; deriving a measure of the user's vision from the matching of the received signals with the displayed numbers; and displaying a result indicative of the measure of the user's vision from the measure.

In certain implementations, the displayed result is in the form of an approximate visual acuity measurement. In certain implementations, the displayed result is in the form of a number of errors.

Another implementation of a visual screening method involves sequentially presenting a plurality of numbers of varying size on a television display; receiving a plurality of signals from a remote controller as a result of a user input from a keypad of the remote controller; determining if the signals received from the remote controller match the displayed numbers; deriving a measure of the user's vision from the matching of the received signals with the displayed numbers; and displaying a result indicative of the measure of the user's vision from the measure.

In certain implementations, the displayed result is in the form of an approximate visual acuity measurement. In certain implementations, the displayed result is in the form of a number of errors. In certain implementations, the sequential presentation is carried out by highlighting numbers selected from a plurality of numbers displayed on the television display. In certain implementations, the numbers are arranged on the display in a grid array having rows and columns In certain implementations, each of the rows of the grid array carries numbers of similar size. In certain implementations, the sequential presentation is carried out by sequentially displaying the plurality of numbers. In certain implementations, the method further involves selecting a vision test program from a television menu system.

Any of the methods described herein can be implemented using a tangible computer readable electronic storage medium storing instructions which, when executed on one or more programmed processors, carry out the method.

A television device consistent with certain implementations that provides visual screening has a television receiver device having a display. A control processor controls at least a portion of the operation of the television receiver device. The processor further carries out a visual screening function involving sequentially presenting a plurality of numbers of varying size for display on the television receiver's display; receiving a plurality of signals from a remote controller as a result of a user input from a keypad of the remote controller; determining if the signals received from the remote controller match the displayed numbers; deriving a measure of the user's vision from the matching of the received signals with the displayed numbers; and causing the television receiver's display to display a result indicative of the measure of the user's vision from the measure.

In certain implementations, the displayed result is in the form of an approximate visual acuity measurement calculated by the processor. In certain implementations, the displayed result is in the form of a number of errors. In certain implementations, the sequential presentation is carried out by highlighting numbers selected from a plurality of numbers displayed on the television display. In certain implementations, the numbers are arranged on the display in a grid array having rows and columns In certain implementations, each of the rows of the grid array carries numbers of similar size. In certain implementations, the sequential presentation is carried out by sequentially displaying the plurality of numbers. In certain implementations, the processor further carries out a process of presenting a vision test program for selection by a user on a television menu system. In certain implementations, the menu system comprises a cross-media bar menu system.

In another implementation of a television device that provides visual screening has a television receiver device having a display. A control processor controls at least a portion of the operation of the television receiver device, said processor further carrying out a visual screening function comprising: presenting a vision test program for selection by a user on a television cross media bar menu system; receiving a user input selecting the vision test program; sequentially presenting a plurality of numbers of varying size for display on the television receiver's display; where the sequential presentation is carried out by highlighting numbers selected from a plurality of numbers displayed on the television display, where the numbers are arranged on the display in a grid array having rows and columns and where each of the rows of the grid array carries numbers of similar size; receiving a plurality of signals from a remote controller as a result of a user input from a keypad of the remote controller; determining if the signals received from the remote controller match the displayed numbers; deriving an approximate visual acuity measure of the user's vision from the matching of the received signals with the displayed numbers; and causing the television receiver's display to display the visual acuity measure.

Those skilled in the art will recognize, upon consideration of the above teachings, that certain of the above exemplary embodiments are based upon use of one or more programmed processors. However, the invention is not limited to such exemplary embodiments, since other embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Certain embodiments described herein, are or may be implemented using one or more programmed processors within a television receiver device executing programming instructions that are broadly described above in flow chart form that can be stored on any suitable tangible electronic or computer readable storage medium. However, those skilled in the art will appreciate, upon consideration of the present teaching, that the processes described above can be implemented in any number of variations and in many suitable programming languages without departing from embodiments of the present invention. For example, the order of certain operations carried out can often be varied, additional operations can be added or operations can be deleted without departing from certain embodiments of the invention. Error trapping can be added and/or enhanced and variations can be made in user interface and information presentation without departing from certain embodiments of the present invention. Such variations are contemplated and considered equivalent.

While certain illustrative embodiments have been described, it is evident that many alternatives, modifications, permutations and variations will become apparent to those skilled in the art in light of the foregoing description.

What is claimed is:

1. A visual screening method, comprising:
on a television display, displaying an array of numbers arranged in rows and columns of numbers, where each of the rows contains numbers of a similar size and where the size of the numbers in each row differs from the size of numbers in each of the other rows;
sequentially highlighting a plurality of the numbers displayed on the television display in order to prompt a user input;
for each of the sequentially highlighted numbers, receiving a signal from a remote controller as a result of a user input to a number pad of the remote controller;
determining if each of the received signals matches the displayed numbers;
deriving a measure of the user's vision from the matching of the received signals with the displayed numbers; and
displaying a result indicative of the measure of the user's vision from the measure.

2. The method according to claim 1, where the displayed result is in the form of an approximate visual acuity measurement.

3. The method according to claim 1, where the displayed result is in the form of a number of errors.

4. A tangible computer readable electronic storage medium storing instructions which, when executed on one or more programmed processors, carry out a method according to claim 1.

5. A visual screening method, comprising:
sequentially presenting a plurality of numbers of varying size on a television display;
receiving a plurality of signals from a remote controller as a result of a user input from a keypad of the remote controller;
determining if the signals received from the remote controller match the displayed numbers;
deriving a measure of the user's vision from the matching of the received signals with the displayed numbers; and
displaying a result indicative of the measure of the user's vision from the measure.

6. The method according to claim 5, where the displayed result is in the form of an approximate visual acuity measurement.

7. The method according to claim 5, where the displayed result is in the form of a number of errors.

8. The method according to claim 5, where the sequential presentation is carried out by highlighting numbers selected from a plurality of numbers displayed on the television display.

9. The method according to claim 8, where the numbers are arranged on the display in a grid array having rows and columns

10. The method according to claim 9, where each of the rows of the grid array carries numbers of similar size.

11. The method according to claim 5, where the sequential presentation is carried out by sequentially displaying the plurality of numbers.

12. The method according to claim 5, further comprising selecting a vision test program from a television menu system.

13. A tangible computer readable electronic storage medium storing instructions which, when executed on one or more programmed processors, carry out a method according to claim 5.

14. A television device that provides visual screening, comprising:
a television receiver device having a display;
a control processor that controls at least a portion of the operation of the television receiver device, said processor further carrying out a visual screening function comprising:
sequentially presenting a plurality of numbers of varying size for display on the television receiver's display;
receiving a plurality of signals from a remote controller as a result of a user input from a keypad of the remote controller;
determining if the signals received from the remote controller match the displayed numbers;
deriving a measure of the user's vision from the matching of the received signals with the displayed numbers; and
causing the television receiver's display to display a result indicative of the measure of the user's vision from the measure.

15. The device according to claim 14, where the displayed result is in the form of an approximate visual acuity measurement calculated by the processor.

16. The device according to claim 14, where the displayed result is in the form of a number of errors.

17. The device according to claim 14, where the sequential presentation is carried out by highlighting numbers selected from a plurality of numbers displayed on the television display.

18. The device according to claim 17, where the numbers are arranged on the display in a grid array having rows and columns

19. The device according to claim 18, where each of the rows of the grid array carries numbers of similar size.

20. The device according to claim 14, where the sequential presentation is carried out by sequentially displaying the plurality of numbers.

21. The device according to claim 14, where the processor further carries out a process of presenting a vision test program for selection by a user on a television menu system.

22. The device according to claim 21, where the menu system comprises a cross-media bar menu system.

23. A television device that provides visual screening, comprising:
a television receiver device having a display;
a control processor that controls at least a portion of the operation of the television receiver device, said processor further carrying out a visual screening function comprising:
presenting a vision test program for selection by a user on a television cross media bar menu system;
receiving a user input selecting the vision test program;
sequentially presenting a plurality of numbers of varying size for display on the television receiver's display;
where the sequential presentation is carried out by highlighting numbers selected from a plurality of numbers displayed on the television display, where the numbers are arranged on the display in a grid array having rows and columns and where each of the rows of the grid array carries numbers of similar size;
receiving a plurality of signals from a remote controller as a result of a user input from a keypad of the remote controller;
determining if the signals received from the remote controller match the displayed numbers;
deriving an approximate visual acuity measure of the user's vision from the matching of the received signals with the displayed numbers; and
causing the television receiver's display to display the visual acuity measure.
